(19) United States
(12) Patent Application Publication
(10) Pub. No.: US 2021/0246399 A1
(43) Pub. Date: Aug. 12, 2021

(54) Polyesterase II

(71) Applicant: Henkel AG & Co. KGaA, Duesseldorf (DE)

(72) Inventors: Christian DEGERING, Erkrath (DE); Thomas HAARMANN, Zwingenberg (DE); Patrick LORENZ, Lorsch (DE); Nina MUSSMANN, Willich (DE); Tina PLOSS, Buettelborn (DE); Martina SCHREITER, Pfungstadt (DE); Ruth SCHWERDTFEGER, Darmstadt (DE); Ren WEI, Borsdorf (DE); Susanne WIELAND, Zons/Dormagen (DE); Wolfgang ZIMMERMANN, Leipzig (DE)

(21) Appl. No.: 16/973,453

(22) PCT Filed: Jun. 25, 2019
(86) PCT No.: PCT/EP2019/066792

§ 371 Date: Dec. 9, 2020

(30) Foreign Application Priority Data
Jun. 28, 2018 (DE) 10 2018 210 609.6

(51) Int. Cl. C11D 3/386; C12N 9/18

(52) U.S. Cl. CPC C11D 3/38636; C12N 9/18

(57) ABSTRACT

An agent, such as a detergent or cleaning agent, may include a polyesterase, as defined herein. A method for cleaning textiles may include applying the agent to a textile for removing soiling. The polyesterase may also help to reduce pilling effects in the agent.

### REFERENCE TO A SEQUENCE LISTING SUBMITTED VIA EFS-WEB

The content of the ASCII text file of the sequence listing named “2018PF35176-Sequence_protocol”, which is 3 kb in size was created on Jun. 28, 2018 and electronically submitted via EFS-Web herewith the application is incorporated by reference in its entirety.

### CROSS-REFERENCE TO RELATED APPLICATIONS

The present application is a national stage entry according to 35 U.S.C. § 371 of PCT application No.: PCT/EP2019/066792 filed on Jun. 25, 2019; which claims priority to German Patent Application Serial No.: 10 2018 210 609.6 filed on Jun. 28, 2018; all of which are incorporated herein by reference in their entirety and for all purposes.

### TECHNICAL FIELD

The field of enzyme technology, in particular the anti-pilling effect of enzymes, such as those used in washing or cleaning agents is described. The invention relates to an agent, in particular a washing or cleaning agent, which contains a polyesterase as defined herein. The present disclosure further relates to a method for cleaning textiles and to the use of the agent for removing stains. Furthermore, the invention is directed to the use of a polyesterase to reduce pilling effects and prevent graying in an agent, such as a washing or cleaning agent.

### BACKGROUND

If washed several times, all types of textiles will pill over time. Pilling refers to the formation of nodules or lint in fabrics. These small pieces of lint are particularly common with short-fiber fabrics. With long-fiber and twisted fibers, however, there is less pilling. Generally, these nodules are caused by loose fibers in the fabric or those that have come loose from the fabric. Due to their smooth surface, synthetic fibers are prone to pilling more than natural fibers, because synthetic fibers can be released from the fabric faster than rough natural fibers. In the case of wool fabrics, these fibers “mat” mainly due to mechanical friction and form nodules on the surface.

The main impact of pilling is an adverse visual effect. Due to the formation of nodules on the surface, fabrics quickly look used and older than they are. In addition, colored textiles appear less brilliant. In contrast, the functionality of the fabric is hardly or not at all impaired. Pilling takes place in particular at places that are subject to high mechanical stress, usually in the shoulder and waist region. Due to the continuous thinning of the material, these stressed regions are particularly at risk of forming holes or even tearing. The undesirable pilling has the consequence that correspondingly impaired textiles are rejected and thrown away by consumers more quickly than would be necessary on the basis of the functionality of the textile.

Furthermore, textiles tend to turn gray when washed. This is because both dirt and detached pigments are released from colored clothes in the washing process. Although attempts are made to keep said dirt and pigments in the washing liquor by means of various washing agent ingredients, it is often not possible to prevent the dirt/pigments from being deposited on the clothing and remaining there. This is the so-called graying effect. This is particularly pronounced for some synthetic fibers such as polyamide, but also polyester.

A technical solution to reduce the pilling effect has so far only been available for cotton textiles. Cellulases are used in the cleaning agent to reduce the pilling effect (DE 69632910 T3). This means that cellulases are used in the washing agent to show anti-pilling or anti-graying effects and thus ensure that clothes look like new for longer. However, cellulases only work on cotton textiles. For other textiles, such as polyester textiles, there is no comparable way to reduce pilling. Therefore, it is desirable and there is a demand for solutions that reduce the pilling of textiles, in particular textiles that contain synthetic fibers such as polyester, in order to keep clothes looking new for as long as possible, i.e. the colors should remain strong, the shape should be preserved and the surfaces should remain smooth and undamaged.

### SUMMARY

Surprisingly, the inventors have found that the polyesterase described herein, which is a hybrid of an LCC cutinase (Sulaiman et al. (2012) “Isolation of a Novel Cutinase Homolog with Polyethylene Terephthalate-Degrading Activity from Leaf-Branch Compost by Using a Metagenomic Approach,” Appl. Environ. Microbiol. 78(5):1556-1562) and a polyesterase from Thermobifida fusca cutinase from Yoshida et al. WP 011291330, is active under washing process conditions and has various nourishing properties for PET textiles. This is surprising insofar as cutinases and PET esterases known to date are more active at higher temperatures (>=60° C.) and, moreover, are only able to degrade PET very slowly. However, the polyesterase used in the present case demonstrates rapid PET degradation at 40° C. It was found that the enzyme prevents pilling on new polyester textiles or facilitates this effect in combination with a cellulase on polyester/cotton blended textiles. In addition, pills that have already been formed can be reduced, i.e. it can produce what is referred to as a “renew” effect. The polyesterase also prevents the graying of white laundry and the fading/graying of colored laundry. It has also been found that, with the appropriate dosage, all of these positive washing properties can be achieved without significantly damaging the fiber. Because the textiles look new longer, they are worn longer and are replaced less quickly. This leads to a reduction in the CO₂ footprint, since less polyester is used.

Therefore, a first aspect is directed to a polyesterase which has at least 70% sequence identity with the amino acid sequence given in SEQ ID NO:1 over its entire length.

In a second aspect, an agent, in particular a washing or cleaning agent, a polyesterase which has at least 70% sequence identity with the amino acid sequence given in SEQ ID NO:1 over its entire length is disclosed.

In a further aspect, methods for cleaning textiles are disclosed, characterized in that an agent is used in at least one method step. The textiles are polyester-containing textiles or consist of polyester.

In another aspect, an agent as described herein, such as a washing or cleaning agent, or a liquid washing agent, may be used for removing stains.

In addition, a further aspect includes the use of the polyesterase described herein for reducing pilling effects and/or increasing the anti-graying effect of an agent, such as a washing or cleaning agent, or a liquid washing agent, the agent containing the polyesterase.

### DETAILED DESCRIPTION

In various embodiments, the polyesterase is a polyesterase which has at least 70% sequence identity with the amino acid sequence given in SEQ ID NO:1 over its entire length. In further embodiments, the polyesterase contained in the agent comprises or substantially consists of or consists of the amino acid sequence given in SEQ ID NO:1. In various embodiments, the invention also includes polyesterases which are derived from the amino acid sequence according to SEQ ID NO:1, for example by means of mutagenesis.

In various embodiments, the polyesterase comprises an amino acid sequence which, over its entire length, is at least 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4% or 99.6% identical to the amino acid sequence given in SEQ ID NO:1 or consists of such a sequence.

In various further embodiments, the polyesterase or the agent containing said polyesterase is characterized in that
(a) the polyesterase is obtainable from a polyesterase as defined above as a starting molecule by single or multiple conservative amino acid substitution; and/or
(b) the polyesterase is obtainable from a polyesterase as defined above as the starting molecule by means of fragmentation or deletion, insertion or substitution mutagenesis, and comprises an amino acid sequence which matches the starting molecule over a length of at least 180, 190, 200, 210, 220, 230, 240, 245, 250, 251, 252, 253, 254, 255, 256, 257, 258, 259 or 260 contiguous amino acids.

The agents contain the polyesterase in an amount of from 0.00001 to 1 wt. %, such as in an amount of from 0.0001 to 0.5 wt. %, or in an amount of from 0.001 to 0.1 wt. %, in each case based on the active protein.

The identity of nucleic acid or amino acid sequences is determined by a sequence comparison. This sequence comparison is based on the BLAST algorithm established and commonly used in the prior art (cf. e.g. Altschul et al. (1990) “Basic local alignment search tool.” J. Mol. Biol. 215:403-410, and Altschul et al. (1997) “Gapped BLAST and PSI-BLAST: a new generation of protein database search programs”; Nucleic Acids Res. 25:3389-3402) and occurs in principle in that similar sequences of nucleotides or amino acids in the nucleic acid or amino acid sequences are assigned to one another. The assignment of the relevant positions shown in a table is referred to as an alignment. Another algorithm available in the prior art is the FASTA algorithm. Sequence comparisons (alignments), in particular multiple sequence comparisons, are created using computer programs. The Clustal series (cf. e.g. Chenna et al. (2003) “Multiple sequence alignment with the Clustal series of programs,” Nucleic Acid Res. 31:3497-3500), T-Coffee (c.f. e.g. Notredame et al. (2000) “T-Coffee: A novel method for multiple sequence alignments,” J. Mol. Biol. 302:205-217) or programs based on these programs or algorithms, for example, are frequently used. Sequence comparisons (alignments) using the computer program Vector NTI® Suite 10.3 (Invitrogen Corporation, 1600 Faraday Avenue, Carlsbad, Calif., USA) with the predetermined, default parameters, and the AlignX module of which for sequence comparisons is based on ClustalW, are also possible.

Such a comparison also allows conclusions to be drawn regarding the similarity of the compared sequences. It is usually given in percent identity, i.e. the proportion of identical nucleotides or amino acid residues in said sequences or in an alignment of corresponding positions. The broader concept of homology takes conserved amino acid exchanges into account in the case of amino acid sequences, i.e. amino acids having similar chemical activity, since they usually perform similar chemical activities within the protein. Therefore, the similarity between the compared sequences can also be expressed in percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small regions. Unless stated otherwise, however, identity or homology information in the present application relates to the entire length of the particular nucleic acid or amino acid sequence indicated.

In various embodiments, the polyesterase comprises an amino acid sequence which is at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4% or 99.6% homologous to the amino acid sequence specified in SEQ ID NO:1 over its entire length.

In a further embodiment, the polyesterase is characterized in that its anti-pilling performance is not significantly reduced compared to that of a polyesterase which comprises an amino acid sequence that corresponds to (or consists of) the amino acid sequence given in SEQ ID NO:1, i.e. has at least 70%, 75%, 80%, 85%, 90%, 95% of the reference anti-pilling performance. This relates in particular to variants which have the sequence identities or homologies given above. The anti-pilling performance can be determined in a washing system which contains a washing agent in a dosage of between 4.5 and 7.0 grams per liter of washing liquor and the polyesterase, the polyesterases to be compared being used in the same concentration (based on active protein) and the anti-pilling performance being determined as described herein. For example, the washing operation can take place for 60 minutes at a temperature of 60° C. and the water can have a water hardness between 15.5 and 16.5° (German hardness). The concentration of the polyesterase in the washing agent intended for this washing system is from 0.00001 to 1 wt. %, such as from 0.0001 to 0.5 wt. %, or from 0.001 to 0.1 wt. %, based on active, purified protein.

A liquid washing agent for such a washing system is composed as follows (all figures in wt. %): 4.4% alkyl benzene sulfonic acid, 5.6% anionic surfactants, 2.4% C₁₂-C₁₈ Na salts of fatty acids, 4.4% non-ionic surfactants, 0.2% phosphonates, 1.4% citric acid, 0.95% NaOH, 0.01% defoamer, 2% glycerol, 0.08% preservatives, 1% ethanol, 1.6% enzyme mix (protease, amylase, cellulase, mannanase) and the remainder being demineralized water. In a non-limiting embodiment, the dosage of the liquid washing agent is between 4.5 and 6.0 grams per liter of washing liquor, for example 4.7, 4.9 or 5.9 grams per liter of washing liquor. Washing takes place in a pH range between pH 8 and pH 10.5, such as between pH 8 and pH 9.

In the context, the anti-pilling performance is determined at 60° C. using a liquid washing agent as indicated above, the washing operation taking place for 60 minutes.

The anti-pilling performance can be tracked using visual matching. In this case, a group of testers assigns the laundry to be examined a value on a scale of 1-5. The value=1 stands for very heavily pilled laundry, while the value=5 is assigned to unpilled laundry.

The activity-equivalent use of the relevant polyesterase ensures that the respective enzymatic properties, for example the anti-pilling performance, are likened even if the ratio of active substance to total protein (the values of the specific activity) diverges. In general, a low specific activity can be compensated for by adding a larger amount of protein.

Proteins can be combined into groups of immunologically related proteins by reaction with an antiserum or a specific antibody. The members of such a group are characterized by the fact that they have the same antigenic determinant recognized by an antibody. They are therefore structurally so similar that they are recognized by an antiserum or certain antibodies. A further object of invention is therefore polyesterases which are characterized by having at least one, two, three or four antigenic determinants matching a polyesterase used in an agent. Due to their immunological similarities, such polyesterases are structurally so similar to the polyesterases used in the agents that a similar function can also be assumed.

Further polyesterases used in the agents can have further amino acid changes, in particular amino acid substitutions, insertions or deletions, compared to the polyesterase described in SEQ ID NO:1. Such polyesterases are, for example, developed by targeted genetic alteration, i.e. by mutagenesis methods, and optimized for specific applications or with regard to specific properties (for example with regard to their catalytic activity, stability, etc.). Furthermore, nucleic acids encoding the polyesterases used can be introduced into recombination approaches and thus used to generate completely new types of polyesterases or other polypeptides.

The aim is to introduce targeted mutations such as substitutions, insertions or deletions into the known molecules in order, for example, to improve the cleaning performance of enzymes. For this purpose, in particular the surface charges and/or the isoelectric point of the molecules and thus their interactions with the substrate can be altered. For instance, the net charge of the enzymes can be altered in order to influence the substrate binding, in particular for use in washing and cleaning agents. Alternatively or in addition, the stability of the polyesterase can be still further increased by one or more corresponding mutations, thereby improving its cleaning performance. Advantageous properties of individual mutations, e.g. individual substitutions, can complement one another. A polyesterase which has already been optimized with regard to specific properties, for example with respect to its activity and/or its anti-pilling performance, can therefore also be developed within the scope.

Another object is therefore a polyesterase, which is characterized in that it is obtainable from a polyesterase as described above as the starting molecule by single or multiple conservative amino acid substitution. The term “conservative amino acid substitution” means the exchange (substitution) of one amino acid residue for another amino acid residue, with this exchange not resulting in a change to the polarity or charge at the position of the exchanged amino acid, e.g. the exchange of a nonpolar amino acid residue for another nonpolar amino acid residue. Conservative amino acid substitutions within the scope include, for example: G=A=S, I=V=L=M, D=E, N=Q, K=R, Y—F, S=T, G=A=I=V=L=M=Y=F=W=P=S=T. The homology of the polyesterases modified in this way to the polyesterase having SEQ ID NO:1 is as defined above.

Alternatively or additionally, the polyesterase is characterized in that it is obtainable from a polyesterase contained in an agent as the starting molecule by fragmentation, deletion, insertion or substitution mutagenesis and comprises an amino acid sequence which matches the starting molecule over a length of at least 180, 190, 200, 210, 220, 230, 240, 245, 250, 251, 252, 253, 254, 255, 256, 257, 258, 259, 260 or 261 contiguous amino acids.

In various embodiments, the polyesterases obtainable in this way also have the sequence identities defined herein of at least 70% with the sequence according to SEQ ID NO:1 even after the mutagenesis/substitution.

For instance, it is possible to delete individual amino acids at the termini or in the loops of the enzyme without the hydrolytic activity being lost or diminished in the process. Furthermore, such fragmentation or deletion, insertion or substitution mutagenesis can also for example reduce the allergenicity of the enzymes concerned and thus improve their overall applicability. Advantageously, the enzymes retain their hydrolytic activity even after mutagenesis, i.e. their hydrolytic activity corresponds at least to that of the starting enzyme, i.e. in a embodiment the hydrolytic activity is at least 80%, such as at least 90% of the activity of the starting enzyme. Other substitutions can also exhibit advantageous effects. Both single and multiple contiguous amino acids can be exchanged for other amino acids.

In various embodiments, the polyesterase can have one or more further amino acids in addition to the sequence N- or C-terminal specified in SEQ ID NO: 1. In certain embodiments, such N-terminal peptides can be the naturally occurring signal peptides for the polyesterase or else a single methionine residue.

An object is an agent which is characterized in that it contains a polyesterase as defined herein. The agent is a washing or cleaning agent.

Unless explicitly indicated otherwise, all percentages that are cited in connection with the compositions/agents described herein relate to wt. %, in each case based on the relevant mixture/the relevant agent.

In the scope, unless otherwise stated, fatty acids and/or fatty alcohols and/or their derivatives represent branched or unbranched carboxylic acids and/or alcohols and/or their derivatives having 6 to 22 carbon atoms. In particular, the oxo-alcohols or their derivatives which can be obtained for example in the Roelen oxosynthesis reaction can be correspondingly used.

Whenever alkaline earth metals are mentioned in the following as counterions for monovalent anions, this means that the alkaline earth metal is naturally only present in half the amount of substance—sufficient to balance the charge—of the anion.

This subject matter covers all conceivable types of washing or cleaning agents, both concentrates and undiluted agents, for use on a commercial scale, in washing machines or for hand washing. These include washing agents for textiles, carpets, or natural fibers, for which the term washing agent is used. In the context, the washing and cleaning agents also include auxiliary washing agents, which are added to the actual washing agent when washing textiles manually or using a machine in order to achieve an additional effect. Furthermore, washing and cleaning agents also include textile pre-treatment and post-treatment agents, i.e. those agents with which the item of laundry is brought into contact before the actual washing cycle, for example to loosen stubborn stains, and also those agents which give the laundry further desirable properties such as a pleasant feel, crease resistance or low static charge in a step subsequent to the actual textile wash. Inter alia, softeners are included in the last-mentioned agents.

The washing or cleaning agents according to invention, which may be in the form of powdered solids, in further-compacted particulate form, homogeneous solutions or suspensions, may contain, in addition to the above-described polyesterase, all known ingredients conventional in such agents, with at least one other ingredient being present in the agent. The agents may in particular contain surfactants, builders, peroxygen compounds or bleach activators. They may also contain water-miscible organic solvents, further enzymes, sequestering agents, electrolytes, pH regulators and/or further auxiliaries such as optical brighteners, graying inhibitors, foam regulators, as well as dyes and fragrances, and combinations thereof.

In particular, a combination of an agent with one or more further ingredients of the agent is advantageous, since, in embodiments, such an agent has improved cleaning performance by virtue of resulting synergisms. In particular, combining an agent with a surfactant and/or a builder and/or a peroxygen compound and/or a bleach activator can result in such a synergism.

Advantageous ingredients of agents are disclosed in international patent application WO 2009/121725, starting at the penultimate paragraph of page 5 and ending after the second paragraph on page 13. Reference is expressly made to this disclosure and the disclosure therein is incorporated in the present patent application.

These and other aspects, features and advantages will become apparent to a person skilled in the art through the study of the following detailed description and claims. Any feature from one aspect can be used in any other aspect. Furthermore, it will readily be understood that the examples contained herein are intended to describe and illustrate but not to limit the invention and that, in particular, the invention is not limited to these examples. Unless indicated otherwise, all percentages indicated are percentages by weight, based on the total weight of the composition. Numerical ranges that are indicated in the format “from x to y” also include the stated values. If several numerical ranges are indicated in this format, it is self-evident that all ranges that result from the combination of the various endpoints are also included.

In addition to the polyesterase, the agents also contain at least one compound from the class of surfactants, in particular selected from anionic and non-ionic, but also cationic, zwitterionic or amphoteric surfactants.

Suitable surfactants are, for example, anionic surfactants of the formula (I)

R—SO₃⁻Y⁺ (I).

In this formula (I), R represents a linear or branched, unsubstituted alkyl aryl functional group. Y represents a monovalent cation or the nth part of an n-valent cation, the alkali metal ions, including Na⁺ or K⁺. Further cations Y⁺ can be selected from NH₄⁺, ½ zn²⁺, ½ Mg²⁺, ½ Ca²⁺, ½ Mn²⁺, and mixtures thereof.

“Alkyl aryl,” as used herein, refers to organic functional groups that consist of an alkyl functional group and an aromatic functional group. Typical examples of functional groups of this kind include, but are not restricted to, alkylbenzene functional groups, such as benzyl, butylbenzene functional groups, nonylbenzene functional groups, decylbenzene functional groups, undecylbenzene functional groups, dodecylbenzene functional groups, tridecylbenzene functional groups and the like.

In different embodiments, surfactants of this kind are selected from linear or branched alkylbenzene sulfonates of the formula A-1

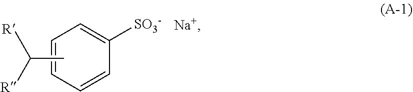

in which R′ and R″ together contain 9 to 19, such as 11 to 15, and in particular 11 to 13, C atoms. A very particularly representative can be described by formula A-la:

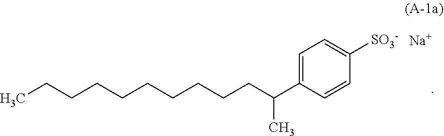

In various embodiments, the compound of the formula (I) is the sodium salt of a linear alkylbenzene sulfonate.

In agents, the at least one compound from the class of anionic surfactants of the formula (I) is contained in an amount of from 0.001 to 30 wt. %, such as 0.001 to 10 wt. %, or 2 to 6 wt. %, or 3 to 5 wt. %, in the washing or cleaning agent, in each case based on the total weight of the cleaning agent.

In various embodiments, the agents contain at least one anionic surfactant of the formula

R¹—O-(AO)ₙ—SO₃⁻X⁺ (II).

In this formula (II), R¹ represents a linear or branched, substituted or unsubstituted alkyl, aryl or alkyl aryl functional group, such as a linear, unsubstituted alkyl functional group, or a fatty alcohol functional group. Non-limiting functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, such as the representatives having an even number of C atoms. Non-limiting functional groups R¹ are derived from C₁₂-C₁₈ fatty alcohols, for example from coconut fatty alcohol, tallow fatty alcohol, lauryl, myristyl, cetyl or stearyl alcohol or from C₁₀-C₂₀ oxo alcohols.

AO represents an ethylene oxide (EO) group or propylene oxide (PO) group, such as an ethylene oxide group. The index n represents an integer of from 1 to 50, such as from 1 to 20, and in particular from 2 to 10. In a non-limiting embodiments, n represents the numbers 2, 3, 4, 5, 6, 7 or 8. X represents a monovalent cation or the nth part of an n-valent cation, in this case the alkali metal ions, which include Na⁺ or K⁺. Further cations X+ can be selected from NH₄⁺, ½ zn²⁺, ½ Mg²⁺, ½ Ca²⁺, ½ Mn²⁺ and mixtures thereof.

In summary, agents in various embodiments thus contain at least one anionic surfactant selected from fatty alcohol ether sulfates of the formula A-2

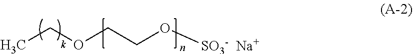

where k=11 to 19, and n=2, 3, 4, 5, 6, 7 or 8. Non-limiting representatives are Na—C₁₂₋₁₄ fatty alcohol ether sulfates having 2 EO (k=11-13, n=2 in formula A-2).

In various embodiments, the cleaning agent contains the at least one anionic surfactant of the formula (II) in an amount of from 2 to 10 wt. %, such as 3 to 8 wt. %, based on the total weight of the cleaning agent.

Other anionic surfactants that can be used are the alkyl sulfates of the formula

R²—O—SO₃⁻X⁺ (III).

In this formula (III), R² represents a linear or branched, substituted or unsubstituted alkyl functional group, such as a linear, unsubstituted alkyl functional group, or a fatty alcohol functional group. Non-limiting functional groups R² are selected from decyl, undecyl, dodecyl, tridecyl, tetradecyl, pentadecyl, hexadecyl, heptadecyl, octadecyl, nonadecyl, eicosyl functional groups and mixtures thereof, such as the representatives having an even number of C atoms. Non-limiting functional groups R² are derived from C₁₂-C₁₈ fatty alcohols, for example from coconut fatty alcohol, tallow fatty alcohol, lauryl, myristyl, cetyl or stearyl alcohol or from C₁₀-C₂₀ oxo alcohols. Y represents a monovalent cation or the nth part of an n-valent cation, in this case the alkali metal ions, which include Na⁺ or K⁺. Further cations Y⁺ can be selected from NH₄⁺, ½ zn²⁺, ½ Mg²⁺, ½ Ca²⁺, ½ Mn²⁺, and mixtures thereof.

In various embodiments, these surfactants are selected from fatty alcohol sulfates of the formula A-3

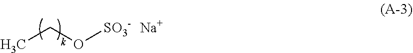

where k=11 to 19. Non-limiting representatives are Na—C₁₂-14 fatty alcohol sulfates (k=11-13 in formula A-3).

In various embodiments, the agent can contain, in addition to the anionic surfactants described above, in particular those of the formulas (I) to (III), or alternatively at least one other surfactant. Other alternative or additional surfactants are, in particular, further anionic surfactants, non-ionic surfactants and mixtures thereof, but also cationic, zwitterionic and amphoteric surfactants.

In various embodiments, the agents comprise at least one non-ionic surfactant, in particular at least one fatty alcohol alkoxylate.

Suitable non-ionic surfactants are those of the formula

R³—O-(AO)ₘ—H (IV),

in which R³ represents a linear or branched, substituted or unsubstituted alkyl functional group, AO is an ethylene oxide (EO) or propylene oxide (PO) group and m is an integer from 1 to 50.

In the aforementioned formula (IV), R³ represents a linear or branched, substituted or unsubstituted alkyl functional group, such as a linear, unsubstituted alkyl functional group, or a fatty alcohol functional group. Non-limiting functional groups R² are selected from decyl, undecyl, dodecyl, tridecyl, tetradecyl, pentadecyl, hexadecyl, heptadecyl, octadecyl, nonadecyl, eicosyl functional groups and mixtures thereof, such as the representatives having an even number of C atoms. Non-limiting functional groups R³ are derived from C₁₂-C₁₈ fatty alcohols, for example from coconut fatty alcohol, tallow fatty alcohol, lauryl, myristyl, cetyl or stearyl alcohol or from C₁₀-C₂₀ oxo alcohols.

AO represents an ethylene oxide (EO) group or propylene oxide (PO) group, such as an ethylene oxide group. The index m represents an integer from 1 to 50, or from 1 to 20, and in particular from 2 to 10. In non-limiting embodiments, m represents the numbers 2, 3, 4, 5, 6, 7 or 8.

In summary, the fatty alcohol alkoxylates to be used are compounds of the formula

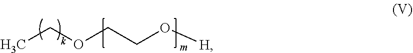

where k=11 to 19, and m=2, 3, 4, 5, 6, 7 or 8. Non-limiting representatives are C₁₂₋₁₈ fatty alcohols having 7 EO (k=11-17, m=7 in formula (V)).

Further non-ionic surfactants which can be contained in the described agents within the meaning include, but are not limited to, alkyl glycosides, alkoxylated alkyl fatty acid esters, amine oxides, fatty acid alkanolamides, hydroxy mixed ethers, sorbitan fatty acid esters, polyhydroxy fatty acid amides and alkoxylated alcohols.

Suitable amphoteric surfactants are, for example, betaines of the formula (Rⁱⁱⁱ)(R^{iv})(R^{v})N⁺CH₂COO⁻, in which Rⁱⁱⁱ denotes an alkyl functional group, which is optionally interrupted by heteroatoms or heteroatom groups, having 8 to 25, such as 10 to 21, carbon atoms, and R^{iv} and R^{v} denote identical or different alkyl functional groups having 1 to 3 carbon atoms, in particular C₁₀-C₁₈ alkyl dimethyl carboxymethyl betaine and C₁₁-C₁₇ alkyl amido propyl dimethyl carboxymethyl betaine.

Suitable cationic surfactants are, inter alia, the quaternary ammonium compounds of the formula (R^{vi})(R^{vii})(R^{viii})(R^{ix})N⁺X⁻, in which R^{vi} to R^{ix} denote four identical or different, and in particular two long-chain and two short-chain, alkyl functional groups, and X⁻ denotes an anion, in particular a halide ion, for example didecyl dimethyl ammonium chloride, alkyl benzyl didecyl ammonium chloride and mixtures thereof. Further suitable cationic surfactants are the quaternary surface-active compounds, in particular having a sulfonium, phosphonium, iodonium or arsonium group, which are also known as antimicrobial washing agents. By using quaternary surface-active compounds having an antimicrobial action, the agent can be designed having an antimicrobial effect or whose antimicrobial effect, which may already be present due to other ingredients, can be improved.

In various embodiments, the total amount of surfactants based on the weight of the agent is 2 to 30 wt. %, such as 5 to 25 wt. %, or 10 to 20 wt. %, or 14 to 18 wt. %, the (linear) alkylbenzene sulfonates being present at most in an amount of from 0.001 to 30 wt. %, such as 0.001 to 10 wt. %, or 2 to 6 wt. %, or 3 to 5 wt. %, based on the weight of the agent.

Washing or cleaning agents can contain further enzymes in addition to the polyesterase. Alternatively, they may also contain other hydrolytic enzymes or other enzymes in a concentration that is expedient for the effectiveness of the agent. One embodiment thus represents agents which comprise one or more enzymes. All enzymes which can develop catalytic activity in the agent, in particular a protease, amylase, cellulase, hemicellulase, mannanase, tannanase, xylanase, xanthanase, xyloglucanase, ß-glucosidase, pectinase, carrageenanase, perhydrolase, oxidase, oxidoreductase or a lipase, and mixtures thereof, can be used as the enzymes. Enzymes are contained in the agent advantageously in each case in an amount of from 1×10⁻⁸ to 5 wt. % based on active protein. Each enzyme is contained in agents in an amount of from 1×10⁻⁷ to 3 wt. %, from 0.00001 to 1 wt. %, from 0.00005 to 0.5 wt. %, from 0.0001 to 0.1 wt. % or from 0.0001 to 0.05 wt. %, based on active protein. In a non-limiting embodiment, the enzymes exhibit synergistic cleaning performance on specific stains or spots, i.e. the enzymes contained in the agent composition support one another in their cleaning performance. Synergistic effects can arise not only between different enzymes, but also between one or more enzymes and other ingredients of the agent.

The amylase(s) is/are an α-amylase. The hemicellulase is a ß-glucanase, a pectinase, a pullulanase and/or a mannanase. The cellulase is a cellulase mixture or a single-component cellulase, such as an endoglucanase and/or a cellobiohydrolase. The oxidoreductase is an oxidase, in particular a choline-oxidase, or a perhydrolase.

The proteases used are alkaline serine proteases. They act as unspecific endopeptidases, i.e. they hydrolyze any acid amide bonds that are inside peptides or proteins and thereby remove protein-containing stains on the item to be cleaned. Their optimum pH is usually in the distinctly alkaline range.

The protein concentration can be determined using known methods, for example the BCA method (bicinchoninic acid; 2,2′-bichinolyl-4,4′-dicarboxylic acid) or the Biuret method. The active protein concentration is determined by titrating the active centers using a suitable irreversible inhibitor (e.g. phenylmethylsulfonylfluoride (PMSF) for proteases) and determining the residual activity (see M. Bender et al. (1966), J. Am. Chem. Soc. 88(24):5890-5913).

In the cleaning agents described herein, the enzymes to be used may furthermore be formulated together with accompanying substances, for example from fermentation. In liquid formulations, the enzymes are used as enzyme liquid formulations.

The enzymes are generally not provided in the form of pure protein, but rather in the form of stabilized, storable and transportable preparations. These pre-formulated preparations include, for example, the solid preparations obtained through granulation, extrusion, or lyophilization or, in particular in the case of liquid or gel agents, solutions of the enzymes, advantageously maximally concentrated, low-water, and/or supplemented with stabilizers or other auxiliaries.

Alternatively, the enzymes can also be encapsulated, for both the solid and the liquid administration form, for example by spray-drying or extrusion of the enzyme solution together with a natural polymer or in the form of capsules, for example those in which the enzymes are enclosed in a set gel, or in those of the core-shell type, in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using inherently known methods, for example by shaking or roll granulation or in fluidized bed processes. Such granules are advantageously low in dust, for example due to the application of polymeric film-formers, and stable in storage due to the coating.

Moreover, it is possible to formulate two or more enzymes together, such that a single granule exhibits a plurality of enzyme activities.

In various embodiments, the agent can have one or more enzyme stabilizers.

Therefore, the agent may further contain an enzyme stabilizer, for example selected from the group consisting of sodium formate, sodium sulfate, lower aliphatic alcohols and boric acid, as well as their esters and salts. Of course, two or more of these compounds can also be used in combination. The salts of the compounds mentioned can also be used in the form of hydrates, such as sodium sulfate decahydrate.

The term “lower aliphatic alcohols” as used herein includes monoalcohols, diols and polyhydric alcohols having up to 6 carbon atoms. In this context, polyols, for example glycerol, (mono)ethylene glycol, (mono)propylene glycol or sorbitol, should be mentioned as belonging to the group of lower aliphatic alcohols, without the claims being restricted thereto.

In addition to the at least one enzyme stabilizer selected from the above group, an agent can also contain at least one further stabilizer. Such stabilizers are known in the prior art.

Reversible protease inhibitors protect the enzymes contained in a washing or cleaning agent from proteolytic degradation by reversibly inhibiting the enzymatic activity of the proteases contained in the agent. Benzamidine hydrochloride, boronic acids or their salts or esters are frequently used as reversible protease inhibitors, including above all derivatives having aromatic groups, for example ortho-, meta- or para-substituted phenylboronic acids, in particular 4-formylphenylboronic acid, or the salts or esters of the mentioned compounds. Peptide aldehydes, that is to say oligopeptides having a reduced C-terminus, in particular those of 2 to 50 monomers, are also used for this purpose. The peptide reversible protease inhibitors include, inter alia, ovomucoid and leupeptin.

Other enzyme stabilizers are amino alcohols such as mono-, di-, triethanol- and -propanolamine and mixtures thereof, aliphatic carboxylic acids up to C₁₂, such as succinic acid, other dicarboxylic acids or salts of the mentioned acids. End-capped fatty acid amide alkoxylates are also suitable for this purpose. Some organic acids used as builders can also stabilize an enzyme. Calcium and/or magnesium salts are also used for this purpose, for example calcium acetate.

Polyamide oligomers or polymeric compounds such as lignin, water-soluble vinyl copolymers or cellulose ethers, acrylic polymers and/or polyamides stabilize the enzyme preparation against physical influences or pH fluctuations, among other things. Polymers containing polyamine N-oxide act simultaneously as enzyme stabilizers and as color transfer inhibitors. Other polymeric stabilizers are linear C₈-C₁₈ polyoxyalkylenes. Alkyl polyglycosides can also stabilize the enzymatic components of the agent and are capable of additionally increasing their performance. Cross-linked N-containing compounds fulfill a double function as soil release agents and as enzyme stabilizers. Hydrophobic, non-ionic polymer stabilizes in particular any cellulase that may be contained.

Reducing agents and antioxidants increase the stability of the enzymes against oxidative decay; for this purpose, sulfur-containing reducing agents are common, such as sodium sulfite and reducing sugars.

In one embodiment, the agents according to are liquid and contain water as the main solvent, i.e. they are aqueous agents. The water content of the aqueous agent is usually 15 to 70 wt. %, such as 20 to 60 wt. %. In various embodiments, the water content is more than 5 wt. %, or more than 15 wt. % or more than 50 wt. %, of water, in each case based on the total amount of agent.

In addition, non-aqueous solvents can be added to the agent. Suitable non-aqueous solvents include monovalent or polyvalent alcohols, alkanol amines or glycol ethers, if they can be mixed with water in the stated concentration range. In a non-limiting embodiment, the solvents are selected from ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, propylene-glycol-t-butylether, di-n-octyl ether and mixtures of these solvents.

The one or more non-aqueous solvents are usually present in an amount of from 0.1 to 10 wt. %, such as 1 to 8 wt. %, based on the total composition.

In addition to the components mentioned so far, the agents can contain other ingredients that further improve the practical and/or aesthetic properties of the cleaning agent. These include, for example, additives for improving the flow and drying behavior, for adjusting the viscosity, and/or for stabilization and other auxiliary and additional substances that are customary in cleaning agents, such as UV stabilizers, perfume, pearlescing agents, dyes, corrosion inhibitors, preservatives, bitterns, organic salts, disinfectants, structuring polymers, defoamers, encapsulated ingredients (e.g. encapsulated perfume), pH adjusters and skin-feel-improving or nourishing additives.

An agent, in particular a washing or cleaning agent, contains at least one water-soluble and/or water-insoluble, organic and/or inorganic builder.

The builders that can generally be used include, in particular, the aminocarboxylic acids and their salts, zeolites, silicates, carbonates, organic (co)builders and—where there are no ecological prejudices against their use—also the phosphates. However, the agents are phosphate-free.

The water-soluble organic builders include polycarboxylic acids, in particular citric acid and saccharic acids, monomeric and polymeric aminopolycarboxylic acids, in particular methylglycinediacetic acid, nitrilotriacetic acid, ethylenediaminetetraacetic acid and polyaspartic acid, polyphosphonic acids, in particular amino tris(methylenephosphonic acid), ethylenediamine tetrakis(methylenephosphonic acid) and 1-hydroxyethane-1,1-diphosphonic acid, polymeric hydroxy compounds such as dextrin, and polymeric (poly)carboxylic acids, polymeric acrylic acids, methacrylic acids, maleic acids, and mixed polymers thereof, which may also contain, in the polymer, small portions of polymerizable substances, without a carboxylic acid functionality. Compounds of this class which are suitable are copolymers of acrylic acid or methacrylic acid with vinyl ethers, such as vinyl methyl ethers, vinyl esters, ethylene, propylene, and styrene, in which the proportion of the acid is at least 50 wt. %. The organic builders may, in particular for the production of liquid agents, be used in the form of aqueous solutions, such as in the form of 30 to 50 wt. % aqueous solutions. All mentioned acids are generally used in the form of their water-soluble salts, in particular their alkali salts.

Organic builders, if desired, can be contained in amounts of up to 40 wt. %, in particular up to 25 wt. %, or from 1 to 8 wt. %. Amounts close to the stated upper limit are used in paste-form or liquid, in particular water-containing, agents. Laundry post-treatment agents, such as softeners, can optionally also be free of organic builders.

Suitable water-soluble inorganic builder materials are, in particular, alkali silicates and, if there are no concerns about their use, also polyphosphates, such as sodium triphosphate. In particular crystalline or amorphous alkali aluminosilicates, if desired, can be used as water-insoluble, water-dispersible inorganic builder materials in amounts of up to 50 wt. %, such as no greater than 40 wt. %, and in liquid agents in particular in amounts of from 1 to 5 wt. %. Among these, crystalline sodium aluminosilicates of washing agent quality, in particular zeolite A, P and optionally X, are non-limiting examples. Amounts close to the stated upper limit are preferably used in solid particulate agents. Suitable aluminosilicates have in particular no particles having a particle size greater than 30 μm and comprise at least 80 wt. % of particles having a size smaller than 10 μm.

Suitable substitutes or partial substitutes for the stated aluminosilicate are crystalline alkali silicates, which may be present alone or in a mixture with amorphous silicates. The alkali silicates that can be used in the agents as builders have a molar ratio of alkali oxide to SiO₂ of less than 0.95, in particular from 1:1.1 to 1:12, and may be present in amorphous or crystalline form. Non-limiting alkali silicates are sodium silicates, in particular amorphous sodium silicates having a Na₂O: SiO₂ molar ratio of from 1:2 to 1:2.8. Non-limiting crystalline silicates, which may be present alone or in a mixture with amorphous silicates, are crystalline phyllosilicates of general formula Na₂SiₓO₂ₓ₊₁.y H₂O, where x, referred to as the module, is a number from 1.9 to 4, y is a number from 0 to 20, and values for x are 2, 3 or 4. Non-limiting crystalline phyllosilicates are those in which x in the stated general formula assumes the values 2 or 3. In particular, both beta-sodium and delta-sodium disilicates (Na₂Si₂O₅.y H₂O) are non-limiting examples. Practically water-free crystalline alkali silicates of the above general formula, in which x is a number from 1.9 to 2.1, which alkali silicates are produced from amorphous alkali silicates, may also be used in agents. In a further embodiment of agents, a crystalline sodium phyllosilicate having a module of from 2 to 3, as can be produced from sand and soda, is used. Crystalline sodium silicates having a module in the range of from 1.9 to 3.5 are used in a further embodiment of agents. If alkali aluminosilicate, in particular zeolite, is also present as an additional builder, the weight ratio of aluminosilicate to silicate, in each case based on water-free active substances, is from 1:10 to 10:1. In agents containing both amorphous and crystalline alkali silicates, the weight ratio of amorphous alkali silicate to crystalline alkali silicate is from 1:2 to 2:1 and in particular from 1:1 to 2:1.

Builders are, if desired, contained in the agents in amounts of up to 60 wt. %, in particular from 5 wt. % to 40 wt. %. Water-soluble builders are optionally in liquid formulations. Laundry post-treatment agents, for example softeners, are free of inorganic builders.

Polymeric thickening agents within the meaning are the polycarboxylates which have a thickening action as polyelectrolytes, such as homo- and copolymerizates of acrylic acid, in particular acrylic acid copolymers such as acrylic acid-methacrylic acid copolymers, and the polysaccharides, in particular heteropolysaccharides, and other conventional thickening polymers.

Suitable polysaccharides or heteropolysaccharides are the polysaccharide gums, for example gum arabic, agar, alginates, carrageenans and their salts, guar, guar gum, tragacanth, gellan, ramsan, dextran or xanthan and their derivatives, for example propoxylated guar, and mixtures thereof. Other polysaccharide thickeners, such as starches or cellulose derivatives, may alternatively or be used in addition to a polysaccharide gum, for example starches of various origins and starch derivatives, for example hydroxyethyl starch, starch phosphate esters or starch acetates, or carboxymethyl cellulose or its sodium salt, methyl, ethyl, hydroxyethyl, hydroxypropyl, hydroxypropylmethyl or hydroxyethylmethyl cellulose or cellulose acetate.

Acrylic acid polymers suitable as polymeric thickening agents are, for example, high-molecular-weight homopolymers of acrylic acid (INCI: carbomer) cross-linked with a polyalkenyl polyether, in particular an allyl ether of sucrose, pentaerythritol or propylene, also referred to as carboxyvinyl polymers.

However, particularly suitable polymeric thickening agents are the following acrylic acid copolymers: (i) copolymers of two or more monomers from the group of acrylic acid, methacrylic acid and their simple esters, such as formed with C₁₋₄ alkanols (INCI: acrylates copolymer) which include, for example, the copolymers of methacrylic acid, butyl acrylate and methyl methacrylate (CAS 25035-69-2) or butyl acrylate and methyl methacrylate (CAS 25852-37-3); (ii) cross-linked high-molecular-weight acrylic acid copolymers, which include for instance the copolymers of C₁₀₋₃₀ alkyl acrylates cross-linked with an allyl ether of sucrose or pentaerythritol with one or more monomers from the group of acrylic acid, methacrylic acid and their simple esters, such as formed by C₁₋₄ alkanols, (INCI: acrylates/C₁₀₋₃₀ alkyl acrylate crosspolymer).

The content of polymeric thickening agent is usually not more than 8 wt. %, such as between 0.1 and 7 wt. %, or between 0.5 and 6 wt. %, in particular between 1 and 5 wt. % or

between 1.5 and 4 wt. %, for example between 2 and 2.5 wt. %, based on the total weight of the agent.

To stabilize the agent, in particular at a high surfactant content, one or more dicarboxylic acids and/or their salts can be added, in particular to a composition of Na salts of adipic, succinic and glutaric acid, for example as is available under the trade name Sokalan® DSC. The use here is advantageously in amounts of 0.1 to 8 wt. %, such as 0.5 to 7 wt. %, in particular 1.3 to 6 wt. % or 2 to 4 wt. %, based on the total weight of the cleaning agent.

However, if the use thereof can be dispensed with, the agent is free of dicarboxylic acids (dicarboxylic acid salts).

The washing agents can be compared with reference washing agents in order to determine the increased anti-pilling performance of the washing agents. A washing system of this kind may be composed as follows (all figures in wt. %): reference agent: 4.4% alkyl benzene sulfonic acid, 5.6% further anionic surfactants, 2.4% C₁₂-C₁₈ Na salts of fatty acids (soaps), 4.4% non-ionic surfactants, 0.2% phosphonates, 1.4% citric acid, 0.95% NaOH, 0.01% defoamer, 2.0% glycerol, 0.08% preservatives, 1% ethanol, 1.6% enzyme mix (protease, amylase, cellulase, mannanase) and the remainder being demineralized water. Agent: 4.4% alkyl benzene sulfonic acid, 5.6% further anionic surfactants, 2.4% C₁₂-C₁₈ Na salts of fatty acids (soaps), 4.4% non-ionic surfactants, 0.2% phosphonates, 1.4% citric acid, 0.95% NaOH, 0.01% defoamer, 2.0% glycerol, 0.08% preservatives, 1% ethanol, 1.6% enzyme mix (protease, amylase, cellulase, mannanase), 0.009% polyesterase and the remainder being demineralized water. In a non-limiting embodiment, the dosage of the liquid washing agent is between 4.5 and 6.0 grams per liter of washing liquor, for example 4.7, 4.9 or 5.9 grams per liter of washing liquor. Washing takes place in a pH range between pH 8 and pH 10.5, such as between pH 8 and pH 9.

The previously mentioned embodiments include all solid, powdered, liquid, gel or pasty administration forms of agents, which may optionally also consist of a plurality of phases and can be present in compressed or uncompressed form. The agent may be present as a flowable powder, in particular having a bulk density of from 300 g/l to 1200 g/l, in particular from 500 g/l to 900 g/l or from 600 g/l to 850 g/l. The solid administration forms of the agent also include extrudates, granules, tablets or pouches. Alternatively, the agent may also be in liquid, gel or pasty form, for example in the form of a non-aqueous liquid washing agent or a non-aqueous paste or in the form of an aqueous liquid washing agent or a water-containing paste. The agent may also be present as a one-component system. Such agents consist of one phase. Alternatively, an agent may also consist of a plurality of phases. Such an agent is therefore divided into a plurality of components (multi-component system).

Another object is a method for the cleaning of textiles, which is characterized in that in at least one method step, a washing agent is used. The textiles contain or consist of polyester.

In various embodiments, the method described above is characterized in that the agent is used at a temperature of from 0 to 100° C., such as 0 to 80° C., or from 30 to 70° C. or from 40 to 60° C.

These include both manual and mechanical methods, such as with mechanical methods. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent. All conceivable washing or cleaning methods can be enhanced in at least one of the method steps by the use of a washing agent or cleaning agent, and therefore represent embodiments. All aspects, objects and embodiments described for the washing agents and cleaning agents are also applicable to this subject matter. Therefore, reference is expressly made at this point to the disclosure at the appropriate point with the note that this disclosure also applies to the above-described methods.

Since enzymes naturally already have catalytic activity and also exhibit this in media which otherwise have no cleaning power, for example in a simple buffer, a single and/or the sole step of such a method can consist in a polyesterase, which is the only cleaning-active component, being brought into contact with the stain, such as in a buffer solution or in water. This constitutes a further embodiment of this subject matter.

Alternative embodiments of this subject matter are also represented by methods for treating textile raw materials or for textile care, in which an agent becomes active in at least one method step. Among these, methods for textile raw materials, such as fibers or textiles with synthetic constituents, and very particularly for those with polyester.

Moreover, the agent described herein, for example as washing or cleaning agents are described above, for the (improved) removal of stains, for example from textiles, in particular polyester textiles.

Finally, the use of a polyesterase may reduce the pilling effects of an agent, such as a washing agent, such as a liquid washing agent, the agent containing the polyesterase. The polyesterase is a polyesterase as defined herein. In various embodiments of the use, the polyesterase is contained in the agent in an amount of from 0.00001 to 1 wt. %, such as in an amount of from 0.0001 to 0.5 wt. %, or in an amount of from 0.001 to 0.1 wt. %. In further various embodiments, the polyesterase, which brings about a reduction in the pilling effect, is applied to textiles, in particular textiles which consist of polyester or comprise polyester.

All aspects, objects and embodiments described for the polyesterases or agents are also applicable to further subjects. Therefore, reference is expressly made at this point to the disclosure at the appropriate point with the note that this disclosure also applies to the above-described agent, the method and the uses.

### EXAMPLES

### Example 1: Expression

A synthetic gene with a nucleotide sequence adapted to the Trichoderma codon usage was used for the expression of the polyesterase. The gene was fused with various secretion signals using Gibson assembly and cloned into a plasmid for amplification in Escherichia coli. This expression plasmid has a strong promoter for the expression of the corresponding mRNA of the polyesterase gene and further elements which allow a selection of Escherichia coli cells which have taken up the expression construct after the transformation.

The corresponding construct for transformation and subsequent integration into the genome of Trichoderma reesei was obtained from this plasmid by restriction with Not I. This transformation fragment contains the elements for the expression of the polyesterase gene and a gene which allows the selection of successfully transformed cells in Trichoderma reesei.

After the most productive expression strain had been selected, the polyesterase was produced in sufficient quantity by fermentation in order to be able to be used for washing application tests.

### Example 2: Wash Test

### Washing Agent Matrix Used

This is a commercially available washing agent matrix that was used for the wash test:

| | Wt. % of active | Wt. % of active |
|---|---|---|
| | substance in the | substance in the |
| Chemical name | raw material | formulation |
| Demineralized water | 100 | Remainder |
| Alkyl benzene sulfonic acid | 96 | 3-7 |
| Anionic surfactants (FAEOS) | 70 | 2-6 |
| C12-C18 fatty acid Na salt | 30 | 0.3-1 |
| Non-ionic surfactants (FAEO) | 100 | 3-7 |
| Phosphonates | 40 | 0.1-0.8 |
| Citric acid | 100 | 0.1-2 |
| NaOH | 50 | 0.3-1 |
| Defoamer | t.q. | 0.005-0.01 |
| Glycerol | 99.5 | 0.3-1 |
| Preservatives | 100 | 0.05-0.1 |
| Boric acid | 100 | 0.3-1 |
| Optical brightener | 90 | 0.01-0.08 |
| Thickener | 25 | 1-3 |
| Enzymes (except polyesterase) | 100 | 0.5-2 |
| Dye, perfume | | |

Dosage 50 mL

### Wash Test to Determine the Anti Pilling Performance of Enzymes

20 identical tests are carried out in succession in a commercially available washing machine. Various polyesters and mixed textiles are used as textiles to be assessed, some of which are new and some of which are pre-pilled. After the 20 tests, the pill reduction of the pre-pilled fabrics and the pill formation of the new fabrics are assessed visually.

The pre-pilled fabrics are produced by washing cycles repeated 20 times at 40° C. in commercially available washing machines.

After each washing cycle, the complete laundry is dried in the dryer.

### Washing Conditions:

Water with 16° dH, 2.5 kg clean filling laundry, 40° C. normal program, 50 ml washing agent as described above per machine

Dosage of the polyesterase to be examined: 50 mg active enzyme per washing machine

Sample 1: only washing agent as described above (comparison reference)

Sample 2: Washing agent+50 mg polyesterase (SEQ ID NO:1)

Result after 20 washes on 100% polyester textile:

Visual sampling of the pills, scale 1-5, very strongly pilled=1, not pilled=5

### Sample 1: 1.6

### Sample 2: 3.4

A change of 0.5 units is considered significant.

The polyesterase significantly improves the pill appearance.

1. A polyesterase having at least 70% sequence identity with the amino acid sequence given in SEQ ID NO:1 over its entire length.

2. The polyesterase according to claim 1, characterized in that the polyesterase comprises an amino acid sequence which, over its entire length, is at least 95% or more identical to the amino acid sequence given in SEQ ID NO:1.

3. The polyesterase according to claim 1, wherein:
the polyesterase comprises one or more single or multiple conservative amino acid substitutions;
the polyesterase comprises a fragmentation, a deletion, an insertion, substitution mutagenesis, or combinations thereof; wherein the amino acid sequence matches the starting molecule over a length of at least 180 contiguous amino acids.

4. An agent comprising:
the polyesterase according to claim 1.

5. The agent according to claim 4, wherein the agent comprises the polyesterase in an amount ranging from 0.00001 to 1 wt. %.

6. The agent according to claim 4, further comprising
at least one additional ingredient selected from the group consisting of surfactants, builders, bleaching agents, bleach activators, water-miscible organic solvents, further enzymes, sequestering agents, electrolytes, pH regulators, optical brighteners, graying inhibitors, foam regulators, dyes, and fragrances, and combinations thereof; and
wherein the agent is present in solid or liquid form.

7. A method for cleaning textiles, wherein the method comprises:
applying an enzyme to one or more textiles; wherein the agent is in accordance with claim 4.

8. The method according to claim 7, wherein the one or more textiles comprise polyester.

9. The method of claim 7, wherein the one or more textiles comprises one or more polyester-containing textiles.

10. The method of claim 7, further comprising reducing pilling effects, increasing the anti-graying effect of the agent, or combinations thereof.

11. The method of claim 7, wherein
the polyesterase comprises an amino acid sequence which, over its entire length, is 95% or more identical to the amino acid sequence given in SEQ ID NO:1.

12. The method of claim 7, wherein:
the polyesterase comprises one or more single or multiple conservative amino acid substitutions;
the polyesterase comprises a fragmentation, a deletion, an insertion, substitution mutagenesis, or combinations thereof; wherein the amino acid sequence matches the starting molecule over a length of at least 180 contiguous amino acids.

13. The method of claim 7, wherein the agent comprises the polyesterase in an amount ranging from 0.00001 to 1 wt. %.

14. The method of claim 7, wherein the agent further comprises at least one additional ingredient selected from the group consisting of surfactants, builders, bleaching agents, bleach activators, water-miscible organic solvents, further enzymes, sequestering agents, electrolytes, pH regulators, optical brighteners, graying inhibitors, foam regulators, dyes and fragrances, and combinations thereof; and wherein the agent is present in solid or liquid form.

15. The agent of claim 4, wherein the agent comprises the polyesterase in an amount ranging from 0.0001 to 0.5 wt. %.

16. The agent of claim 4, wherein the agent is a washing or cleaning agent.


SEQUENCE LISTING (1 sequence)

SEQ ID NO: 1 - protein, 261 aa, Artificial
  Hybrid polyesterase
ANPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAIPLTPWHLNKNWSSVTVPTLIIGADLDTIAPVSQHAIPFYQNLPSTTPKVYVELDNASHFAPNSNNAAISVYTISWMKLWVDNDTRYRQFLCNVNDPALSDFRTNNRHCQLE